SEQUENCE LISTING (19 sequences)

SEQ ID NO: 1 - protein, 8 aa, unidentified
  REGION 1..8: note = Synthetic
GYTFTSYY

SEQ ID NO: 2 - protein, 8 aa, unidentified
  REGION 1..8: note = Synthetic
INPSNAGT

SEQ ID NO: 3 - protein, 12 aa, unidentified
  REGION 1..12: note = Synthetic
TREGNPYYTMNY

SEQ ID NO: 4 - protein, 12 aa, unidentified
  REGION 1..12: note = Synthetic
QSLLNSGNQKTY

SEQ ID NO: 5 - skipped

SEQ ID NO: 6 - protein, 9 aa, unidentified
  REGION 1..9: note = Synthetic
QNDHSYPFT

SEQ ID NO: 7 - protein, 119 aa, unidentified
  REGION 1..119: note = Synthetic
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSS

SEQ ID NO: 8 - protein, 113 aa, unidentified
  REGION 1..113: note = Synthetic
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIK

SEQ ID NO: 9 - protein, 449 aa, unidentified
  REGION 1..449: note = Synthetic
  REGION 1..119: note = variable domain
  REGION 120..449: note = ConstantRegion
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 220 aa, unidentified
  REGION 1..220: note = Synthetic
  REGION 1..119: note = VariableRegion
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, unidentified
  REGION 1..446: note = Synthetic
  REGION 1..119: note = VariableRegion
  REGION 120..446: note = ConstantRegion
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 446 aa, unidentified
  REGION 1..446: note = Synthetic
  REGION 1..119: note = VariableDomain
  REGION 120..446: note = ConstantRegion
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 449 aa, unidentified
  REGION 1..449: note = Synthetic
  REGION 1..119: note = VariableDomain
  REGION 120..449: note = ConstantRegion
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 449 aa, unidentified
  REGION 1..446: note = Synthetic
  REGION 1..119: note = VariableDomain
  REGION 120..446: note = ConstantRegion
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 446 aa, unidentified
  REGION 1..446: note = Synthetic
  REGION 1..119: note = VariableDomain
  REGION 120..446: note = ConstantRegion
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 446 aa, unidentified
  REGION 1..446: note = Synthetic
  REGION 1..119: note = VariableDomain
  REGION 120..446: note = ConstantRegion
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 446 aa, unidentified
  REGION 1..446: note = Synthetic
  REGION 1..119: note = VariableDomain
  REGION 120..446: note = ConstantRegion
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 446 aa, unidentified
  REGION 1..446: note = Synthetic
  REGION 1..119: note = VariableDomain
  REGION 120..446: note = ConstantRegion
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 10 aa, unidentified
  REGION 1..10: note = Synthetic
FLSRPTEKTI

(19) United States
(12) Patent Application Publication
(10) Pub. No.: US 2025/0002569 A1
(43) Pub. Date: Jan. 2, 2025

(54) METHOD OF TREATING OSTEOARTHRITIS

(71) Applicant: AlaMab Therapeutics, Inc., Princeton, NJ (US)

(72) Inventor: Yanfeng Zhang, Phoenixville, PA (US)

(21) Appl. No.: 18/757,078

(22) Filed: Jun. 27, 2024

Related U.S. Application Data
(60) Provisional application No. 63/511,563, filed on Jun. 30, 2023.

(51) Int. Cl. C07K 16/18; A61K 9/00; A61K 39/00; A61P 19/02

(52) U.S. Cl. CPC C07K 16/18; A61K 9/0019; A61P 19/02; A61K 2039/505; C07K 2317/565; C07K 2317/76

(57) ABSTRACT

The present disclosure provides compositions and methods of treating osteoarthritis such as osteoarthritis in a subject. The method comprises blocking the opening of Cx43 hemichannel in chondrocytes by, e.g., using a composition comprising an anti-connexin 43 antibody.

### CROSS-REFERENCE TO RELATED APPLICATION

This application claims priority to U.S. Provisional Application No. 63/511,563, filed on Jun. 30, 2023, the disclosure of which is hereby incorporated by reference in its entirety.

### INCORPORATION BY REFERENCE OF SEQUENCE LISTING

The Sequence Listing titled 172628-201002_US_SL.xml, which was created on Jun. 19, 2024 and is 34,858 bytes in size, is hereby incorporated by reference in its entirety.

### BACKGROUND

Osteoarthritis (OA) is a prevalent type of arthritis that affects approximately 20% of U.S. adults. Osteoarthritis causes the degeneration of joints including articular cartilage and subchondral bone, and is characterized by a loss of articular cartilage leading to narrowing of joint space, increased joint friction and potential structure remodeling. Current treatment includes exercise, lifestyle change and analgesics. If symptom becomes severe, joint replacement surgery is normally performed. However, most existing therapies fail to address the underlying cause of osteoarthritis (i.e., the deterioration of cartilage).

Chondrocytes express connexin Cx43 hemichannels, and these channels mediate the passage of small molecules (less than 1.2 kDa) between inside/outside of the cell. Hemichannels are normally closed, but under certain conditions, such as mechanical stress and inflammatory conditions, they are activated and opened. Opened Cx43 hemichannels in chondrocytes promote inflammatory response with the release of pro-inflammatory factors, such as prostaglandin E2 (PGE2) and ATP. Inhibiting the opening of Cx43 hemichannels in chondrocytes (e.g., by chemical reagents, etc.), can suppress the inflammation and the development of osteoarthritis.

Thus, there remains a need for therapies for effective treatment of osteoarthritis and other pathological conditions associated with increased hemichannel activity.

### SUMMARY

In some aspects of the disclosure, provided herein are methods for treating osteoarthritis in a subject in need thereof, comprising administering to the subject at least one dose of an anti-connexin 43 (Cx43) antibody. In some embodiments, the anti-Cx43 antibody comprises heavy chain CDR sequences and light chain CDR sequences as follows:

HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5;
and
LCDR3: SEQ ID NO: 6.

In some embodiments, the method comprises administering a first dose of the anti-Cx43 antibody at day 1, and a second dose at day 8. In some embodiments, the method further comprises administering a third dose of the anti-Cx43 antibody at day 15. In some embodiments, the method further comprises administering a fourth dose of the anti-Cx43 antibody at day 22. In some embodiments, the method further comprises administering subsequently doses weekly after the fourth dose.

In other aspects of the disclosure, provided herein are methods for treating osteoarthritis in a subject in need thereof, comprising administering to the subject an effective amount of an anti-connexin 43 (Cx43) antibody, wherein the anti-Cx43 antibody is administered according to the following dosage regimen:
i) a first dose at day 1;
ii) a second dose at day 8;
iii) a third dose at day 15; and
iv) a fourth dose at day 21.

In some embodiments, the method further comprises administering subsequent doses of the anti-Cx43 antibody weekly after the fourth dose.

In some embodiments, the anti-Cx43 antibody comprises heavy chain CDR sequences and light chain sequences as follows:

HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5;
and
LCDR3: SEQ ID NO: 6.

In some embodiments of any of the methods of the disclosure, the first, second, third, fourth and/or subsequent doses are about 0.01 mg/kg to about 100 mg/kg. In some embodiments, the first, second, third, fourth and/or subsequent doses are about 15 mg/kg. In some embodiments, the first, second, third, fourth and/or subsequent doses are about 25 mg/kg. In some embodiments, the first, second, third, fourth and/or subsequent doses are about 50 mg/kg.

In some embodiments of any of the methods of the disclosure, the anti-Cx43 antibody administered intravenously.

In some embodiments of any of the methods of the disclosure, at least one indicator of osteoarthritis severity is assessed at least one day to one week after each dose is administered. In some embodiments, the at least one indicator of osteoarthritis severity comprises: (a) pain threshold of the arthritis, (b) gait score, or (c) weight bearing difference. In some embodiments, at least one indicator of osteoarthritis severity is improved after at least one dose of the anti-Cx43 antibody is administered.

In some embodiments of any of the methods of the disclosure, the anti-Cx43 antibody comprises heavy chain variable sequence of SEQ ID NO: 7, and/or light chain variable sequence of SEQ ID NO: 8. In some embodiments of any of the methods of the disclosure, the anti-Cx43 antibody comprises heavy chain sequence of any one of SEQ ID NOs: 9 and 11-18, and/or light chain sequence of SEQ ID NO: 10. In some embodiments, the anti-Cx43 antibody comprises heavy chain sequence of SEQ ID NO: 9, and/or light chain sequence of SEQ ID NO: 10.

In some embodiments of any of the methods of the disclosure, the anti-Cx43 antibody blocks the opening of Cx43 hemichannel in the subject.

In some embodiments of any of the methods of the disclosure, the subject is a human.

### BRIEF DESCRIPTION OF DRAWINGS

FIG. 1 depicts the effect of the effect of treatment with an anti-Cx43 antibody on the gait score (paw pressure score) of an osteoarthritis rat model. The osteoarthritis rat model was established by partial medial meniscectomy in combination with anterior cruciate ligament transection (pMMx+ACLT). The anti-Cx43 antibody was administered intravenously, once a week, and four times in total, starting at days 15, 22, 29 and 36 post-osteoarthritis model establishment (corresponding to days 1, 8, 15 and 22 of treatment). Three different dosages of the anti-Cx43 antibody were tested (15 mg/kg, 25 mg/kg, and 50 mg/kg). Duloxetine was used as a control substance and was administered orally daily at a dose of 5 mg/kg. The gait score (paw pressure score) was measured at days 16, 23, 30, and 37 post-osteoarthritis model establishment. Each data point corresponds to the mean gait score (±standard error of the mean [SEM], n=8). As used in the figure: Control, control rats; Model, osteoarthritis rat model (vehicle-treated); and AM1, anti-Cx43 antibody ALMB-0166.

FIG. 2 depicts the effect of treatment with an anti-Cx43 antibody on the pain threshold of plantar pain of an osteoarthritis rat model. The osteoarthritis rat model was established by pMMx+ACLT. The anti-Cx43 antibody was administered intravenously, once a week, and four times in total, starting at days 15, 22, 29 and 36 post-osteoarthritis model establishment (corresponding to days 1, 8, 15 and 22 of treatment). Three different dosages of the anti-Cx43 antibody were tested (15 mg/kg, 25 mg/kg, and 50 mg/kg). Duloxetine was used as a control substance and was administered orally daily at a dose of 5 mg/kg. The pain threshold of plantar pain was measured at days 1, 15, 16, 23, 30, and 37 post-osteoarthritis model establishment. Each data point corresponds to the mean gait score (±SEM, n=8). As used in the figure: Control, control rats; Model, osteoarthritis rat model (vehicle-treated); AM1, anti-Cx43 antibody ALMB-0166; and 50% MWT, positive reaction in 50% chance under stimulus intensity.

FIGS. 3A-3D depict the effect of the effect of treatment with an anti-Cx43 antibody on the pain threshold of plantar pain of an osteoarthritis rat model. The osteoarthritis rat model was established by pMMx+ACLT. The anti-Cx43 antibody was administered intravenously, once a week, and four times in total, starting at days 15, 22, 29 and 36 post-osteoarthritis model establishment (corresponding to days 1, 8, 15 and 22 of treatment). Three different dosages of the anti-Cx43 antibody were tested (15 mg/kg, 25 mg/kg, and 50 mg/kg). Duloxetine was used as a control substance and was administered orally daily at a dose of 5 mg/kg. The figures show the pain threshold of plantar pain at day 16 (FIG. 3A), day 23 (FIG. 3B), day 30 (FIG. 3C), and day 37 (FIG. 3D) following osteoarthritis model establishment. Each data point corresponds to the mean gait score (±SEM, n=8). As used in the figure: Control, control rats; Model, osteoarthritis rat model (vehicle-treated); AM1, anti-Cx43 antibody ALMB-0166; 50% MWT, positive reaction in 50% chance under stimulus intensity; ##, p<0.01 as compared to control rats; *, p<0.05 as compared to osteoarthritis model rat; **, p<0.01 as compared to osteoarthritis model rat.

FIG. 4 depicts the effect of the effect of treatment with an anti-Cx43 antibody on the wight bearing difference of hind legs of an osteoarthritis rat model. The osteoarthritis rat model was established by pMMx+ACLT. The anti-Cx43 antibody was administered intravenously, once a week, and four times in total, starting at days 15, 22, 29 and 36 post-osteoarthritis model establishment (corresponding to days 1, 8, 15 and 22 of treatment). Three different dosages of the anti-Cx43 antibody were tested (15 mg/kg, 25 mg/kg, and 50 mg/kg). Duloxetine was used as a control substance and was administered orally daily at a dose of 5 mg/kg. The weight bearing difference of hind legs was measured at days 1, 15, 16, 23, 30, and 37 post-osteoarthritis model establishment. Each data point corresponds to the mean gait score (±SEM, n=8). As used in the figure: Control, control rats; Model, osteoarthritis rat model (vehicle-treated); and AM1, anti-Cx43 antibody ALMB-0166.

FIGS. 5A-5D depict the effect of the effect of treatment with an anti-Cx43 antibody on the weight bearing difference of an osteoarthritis rat model. The osteoarthritis rat model was established by pMMx+ACLT. The anti-Cx43 antibody was administered intravenously, once a week, and four times in total, starting at days 15, 22, 29 and 36 post-osteoarthritis model establishment (corresponding to days 1, 8, 15 and 22 of treatment). Three different dosages of the anti-Cx43 antibody were tested (15 mg/kg, 25 mg/kg, and 50 mg/kg). Duloxetine was used as a control substance and was administered orally daily at a dose of 5 mg/kg. The figures show the weight bearing difference of hind legs at day 16 (FIG. 5A), day 23 (FIG. 5B), day 30 (FIG. 5C), and day 37 (FIG. 5D) following osteoarthritis model establishment. Each data point corresponds to the mean gait score (±SEM, n=8). As used in the figure: Control, control rats; Model, osteoarthritis rat model (vehicle-treated); AM1, anti-Cx43 antibody ALMB-0166; ##, p<0.01 as compared to control rats; **, p<0.01 as compared to osteoarthritis model rat.

### DETAILED DESCRIPTION

The present disclosure provides methods and compositions for treating osteoarthritis in a subject or patent in need thereof, comprising administering at least one dose of an anti-Cx43 antibody to the subject or patent. In some embodiments, the anti-Cx43 antibody blocks or inhibits the opening of Cx43 hemichannel. In some embodiments, the anti-Cx43 antibody comprises specific CDR amino acid sequences. In some embodiments, the anti-Cx43 antibody is administered according to a dosing regimen.

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “providing” is used according to its ordinary meaning to supply or furnish for use. In some embodiments, the protein (e.g., an antibody) is provided directly by administering the protein, while in other embodiments, the protein (e.g., an antibody) is effectively provided by administering a nucleic acid that encodes the protein. In certain aspects the invention contemplates compositions comprising various combinations of nucleic acid, antigens, peptides, and/or epitopes.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “percent sequence identity” or “sequence identity” refers to the degree of identity between any given query sequence and a subject sequence. A percent identity for any query nucleic acid or amino acid sequence, relative to another subject nucleic acid or amino acid sequence can be determined using tools and technologies known in the art, for example, NCBI BLAST.

The term “pharmaceutical formulation” refers to a preparation that contains a therapeutic agent (e.g., an anti-Cx43 antibody). In such form as to permit the biological activity of the antibody to be effective, and which contains no additional components which are unacceptably toxic to a subject to which the formulation would be administered.

As used herein, the term “antigen” is a molecule capable of being bound by an antibody or T-cell receptor. In certain embodiments, binding moieties other than antibodies are engineered to specifically bind to an antigen, e.g., aptamers, avimers, and the like.

As used herein, the term “specifically binds” is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹. Antibodies may, for example, bind with an EC so of 50 nM or less, 10 nM or less, 1 nM or less, 100 pM or less, or more preferably 10 pM or less. As kwon in the art, a variety of immunoassay formats may be used to select antibodies specifically immunoreactive with an antigen. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

An “affinity matured” antibody has one or more alterations in one or more hypervariable regions thereof which result an improvement in the affinity of the antibody for antigen, compared to a parent antibody which does not possess those alteration(s). In one aspect, affinity matured antibodies will have nanomolar or even picomolar affinities for the target antigen. Affinity matured antibodies are produced by procedures known in the art. Marks et al., Biotechnology, 10:779-783 (1992) describes affinity maturation by VH and VL domain shuffling. Random mutagenesis of CDR and/or framework residues is described by: Barbas et al., Proc Nat. Acad. Sci, USA, 91:3809-3813 (1994); Schier et al., Gene, 169:147-155 (1995); Yelton et al., J. Immunol., 155:1994-2004 (1995); Jackson et al., J. Immunol., 154 (7): 3310-9 (1995); and Hawkins et al., J. Mol. Biol., 226:889-896 (1992).

“Arthritis” is used herein to refer to any disorder that affects joints. Symptoms generally include one or more of joint pain, stiffness, tenderness, redness, warmth, swelling, and decreased range of motion of the affected joints. In some types of arthritis, other organs are also affected. Onset of an arthritis in a subject can be gradual or sudden (e.g., resulting from an injury or infection). Exemplary types of arthritis include, but are not limited to, osteoarthritis, rheumatoid arthritis, gout, septic arthritis, psoriatic arthritis, ankylosing spondylitis, juvenile idiopathic arthritis and Still's disease. An arthritis may be a secondary disorder resulting from another primary disease, such as Lyme disease, systemic lupus erythematosus, or Ehlers-Danlos syndrome.

“Osteoarthritis” as used herein, refers to a type of arthritis that involves wear-and-tear damage to a joint's cartilage. Osteoarthritis results from the deterioration of joint cartilage, which is composed of specialized cells known as chondrocytes. Chondrocytes produce a collagenous extracellular matrix containing collagen and proteoglycans. The loss or breakdown of cartilage increases friction between the bones of the joint, which can lead to the growth of bone spurs (osteophytes) or the release of cartilage and bone fragments into the joint space. Symptoms of osteoarthritis include joint pain, stiffness, reduced range of motion, joint swelling and weakness or numbness of the arms and legs. Osteoarthritis may develop in any joint, but it most commonly affects hands and fingers, wrists, knees, hips, feet, neck and back. The development of osteoarthritis may be due to age (e.g., resulting from wear and tear of cartilage over many years), or it can be induced by a joint injury or infection.

The term “affinity” refers to the strength of a binding reaction between a binding domain of an antibody and an epitope. It is the sum of the attractive and repulsive forces operating between the binding domain and the epitope. The term affinity, as used herein, refers to the dissociation constant KD.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. In one embodiment, an epitope is a region of an antigen that is bound by an antibody. In certain embodiments, an antibody is said to specifically bind an antigen when it preferentially recognizes its target antigen in a complex mixture of proteins and/or macromolecules. Methods for epitope mapping are well known in the art, such as X-ray co-crystallography, array-based oligo-peptide scanning, site-directed mutagenesis, high throughput mutagenesis mapping and hydrogen-deuterium exchange. Epitopes can be formed both from contiguous amino acids, or noncontiguous amino acids juxtaposed by tertiary folding of a protein. Epitopes formed from contiguous ammo acid are typically retained on exposure to denaturing solvents, whereas epitopes formed by tertiary folding are typically lost on treatment with denaturing solvents. An epitope typically includes at least 3, and more usually, at least 5 or 8-10 amino acid in a unique spatial conformation.

The terms “reduce,” “inhibit” and “block” as used interchangeably herein, refer to any statistically significant decrease in biological activity (e.g., hemichannel opening). For example, “reduction” can refer to a decrease of about 10%, 20%, 30%, 40%, 50%, 60%, 70%, 80%, 90%, or 100% in biological activity.

### II. Antibodies

The term “antibody” or “antibodies” herein is used in the broadest sense and specifically covers full length antibody, antibody peptide(s) or immunoglobulin(s), monoclonal antibodies, chimeric antibodies, polyclonal antibodies, human antibodies, humanized antibodies and antibodies from non-human species, including human antibodies derived from a human germline immunoglobulin sequence transduced into the non-human species, e.g., mouse, sheep, chicken or goat, recombinant antigen binding forms such as monobodies and diabodies, multi-specific antibodies (e.g., bispecific antibodies), and individual antigen binding fragments of any of the foregoing, e.g., of an antibody or the antibody from which it is derived, including dAbs, Fv, scFv, Fab, F(ab)′2, Fab′.

Antibody-like binding peptidomimetics are also contemplated in the methods described herein. Liu et al. (2003) describe “antibody-like binding peptidomimetics” (ABiPs), which are peptides that act as pared-down antibodies and have certain advantages of longer serum half-life as well as less cumbersome synthesis methods.

The term “monoclonal antibody” as used herein refers to an antibody obtained from a population of substantially homogeneous antibodies, i.e., the individual antibodies comprising the population are identical and/or bind the same epitope. The modifier “monoclonal” indicates the character of the antibody as being obtained from a substantially homogeneous population of antibodies, and is not to be construed as requiring production of the antibody by any particular method.

“Antigen binding fragments” of an antibody preferably comprise at least the variable regions of the heavy and/or light chains of an anti-Cx43 antibody. For example, an antigen binding fragment of anti-Cx43 antibodies can comprise amino acid sequences of SEQ ID NOs: 7 and 8. Examples of such antigen binding fragments include Fab fragments, Fab′ fragments, Fv fragments, scFv and F(ab′)2 fragments. Antigen binding fragments of an antibody can be produced by enzymatic cleavage or by recombinant techniques. For instance, papain or pepsin cleavage can be used to generate Fab or F(ab′)2 fragments, respectively. Antibodies can also be produced in a variety of truncated forms using antibody genes in which one or more stop codons have been introduced upstream of the natural stop site. For example, a recombinant construct encoding the heavy chain of an F(ab′)₂ fragment can be designed to include DNA sequences encoding the CH1 domain and hinge region of the heavy chain. In one aspect, antigen binding fragments blocks or inhibits the opening of Cx43 hemi-channel in a subject and the effects associated with opening of Cx43 hemi-channel.

A “multi-specific antibody” as used herein refers to an artificial antibody that has two or more different portions, with each portion including the antigen binding region of an antibody to a different antigen or epitope. The portions of the multi-specific antibody can be, for example, full-length antibodies or antibody binding fragments. Bispecific or bifunctional antibodies are multi-specific antibodies that have two different portions (e.g., two different heavy and light chain pairs, or two different antibody binding fragments) that bind two different antigens or epitopes. Multi-specific antibodies can be produced by a variety of methods, including fusion of hybridomas or ligation of antibody binding fragments. For example, Songsivilai and Lachmann, Clin Exp Immunol, 79:315-21 (1990); Kostelny et al., J. Immunol., 148:1547-53 (1992). In some cases, a multi-specific antibody (e.g., a bispecific antibody) includes at least one portion an anti-Cx43 antibody that blocks or inhibits the opening of Cx43 hemi-channel in a subject and the effects associated with opening of Cx43 hemi-channel.

A “therapeutic monoclonal antibody” is an antibody used for therapy of a human subject. Therapeutic monoclonal antibodies disclosed herein include anti-Cx43 antibodies. Antibody “effector functions” refer to those biological activities attributable to the Fc region (a native sequence Fc region or amino acid sequence variant Fc region) of an antibody. Examples of antibody effector functions include C1q binding, complement dependent cytotoxicity, Fc receptor binding, antibody-dependent cell-mediated cytotoxicity (ADCC), phagocytosis, down regulation of cell surface receptors (e.g., B cell receptor; BCR), and the like. To assess ADCC activity of a molecule of interest, an in vitro ADCC assay, such as those described in U.S. Pat. Nos. 5,500,362 or 5,821,337 may be performed.

An anti-Cx43 antibody of the disclosure can also be conjugated to at least one agent or moiety to form an antibody conjugate. The antibody may be linked to the agent covalently or non-covalently. The agent or moiety can increase the diagnostic or therapeutic potential of the antibody, and include, but are not limited to, effector or reporter molecules. Effector molecules include molecules with a desired activity (e.g., cytotoxic activity). Non-limiting examples of effector molecules that can be attached to an antibody include toxins, small molecule drugs, therapeutic enzymes, cytokines, antibiotics, radiolabeled nucleotides, and the like. Reporter molecules are molecules that can be detected by an assay, and include, without limitation, enzymes, radioactive labels, haptens, fluorescent labels, phosphorescent molecules, chemically luminescent molecules, chromophores, luminescent molecules, photoaffinity molecules, colored particles, or ligands (e.g., biotin).

Depending on the amino acid sequence of the constant domain of their heavy chains, full length antibodies can be assigned to different “classes.” There are five major classes of full-length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ and μ, respectively. The subunit structures and three-dimensional configurations of different classes of antibodies are well known. The “light chains” of antibodies from any vertebrate species can be assigned to one of two clearly distinct types, called kappa (κ) and lambda (λ), based on the amino acid sequences of their constant domains.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “hypervariable region” when used herein refers to the amino acid residues of an antibody which are responsible for antigen binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol., 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein.

As will be appreciated by those in the art, the CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

Amino acid substitutions are typically of single residues; insertions usually will be on the order of from about one to about twenty amino acid residues, although considerably larger insertions may be tolerated. Deletions range from about one to about twenty amino acid residues, although in some cases deletions may be much larger.

Substitutions, deletions, insertions or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances.

The term “Fab” or “Fab region,” as used herein, is meant the polypeptide that comprises the VH, CH1, VL, and CL immunoglobulin domains. Fab may refer to this region in isolation, or this region in the context of a full-length antibody, antibody fragment or Fab fusion protein, or any other antibody embodiments as outlined herein.

The term “Fv,” “Fv fragment” or “Fv region,” as used herein, refers to a polypeptide that comprises the VL and VH domains of a single antibody.

The term “framework,” as used herein, is refers to the region of an antibody variable domain exclusive of those regions defined as CDRs. Each antibody variable domain framework can be further subdivided into the contiguous regions separated by the CDRs (FR1, FR2, FR3 and FR4).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. For further details, see Jones et al., Nature, 321:522-525 (1986); Riechmann et al., Nature, 332:323-329 (1988); and Presta, Curr. Op. Struct. Biol., 2:593-596 (1992).

An “isolated” antibody is one which has been identified and separated and/or recovered from a component of its natural environment. In certain embodiments, the antibody will be purified (1) to greater than 95% by weight of protein as determined by the Lowry method, and alternatively, more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

### Anti-Cx43 Antibodies

Certain aspects of the present disclosure provide compositions and methods of treating osteoarthritis in a subject. The methods and compositions comprise an anti-Cx43 antibody which specifically binds Cx43 hemichannel and blocks or inhibits the opening of it in chondrocytes.

The anti-Cx43 antibody can be any antibody specifically binds Cx43 known in the art. In various embodiments, the anti-Cx43 antibody used in the methods comprises an HCDR1 amino acid sequence having at least 90%, at least 95%, or at least 99% sequence identity to SEQ ID NO: 1, an HCDR2 amino acid sequence having at least 90%, at least 95%, or at least 99% sequence identity to SEQ ID NO: 2, and an HCDR3 amino acid sequence having at least 90%, at least 95%, or at least 99% sequence identity to SEQ ID NO: 3; and/or a LCDR1 amino acid sequence having at least 90%, at least 95%, or at least 99% sequence identity to SEQ ID NO: 4, a LCDR2 amino acid sequence having at least 90%, at least 95%, or at least 99% sequence identity to SEQ ID NO: 5, a LCDR3 amino acid sequence having at least 90%, at least 95%, or at least 99% sequence identity to SEQ ID NO: 6. In some specific embodiments, the anti-Cx43 antibody comprises an HCDR1 amino acid sequence identical to SEQ ID NO: 1, an HCDR2 amino acid sequence identical to SEQ ID NO: 2, and an HCDR3 amino acid sequence identical to SEQ ID NO: 3; and/or a LCDR1 amino acid sequence identical to SEQ ID NO: 4, a LCDR1 amino acid sequence identical to SEQ ID NO: 5, and a LCDR3 amino acid sequence identical to SEQ ID NO: 6.

In various embodiments, the anti-Cx43 antibody used in the method comprise a heavy chain having at least 80%, at least 85%, at least 90%, at least 95%, or at least 99% sequence identity to any one of SEQ ID NOs: 9 and 11-18; and/or a light chain having at least 80%, at least 85%, at least 90%, at least 95%, or at least 99% sequence identity to any one of SEQ ID NO: 10. In some specific embodiments, the anti-Cx43 antibody comprise a heavy chain having at least 80%, at least 85%, at least 90%, at least 95%, or at least 99% sequence identity to SEQ ID NO: 9; and a light chain having at least 80%, at least 85%, at least 90%, at least 95%, or at least 99% sequence identity to any one of SEQ ID NO: 10. In some other specific embodiments, the anti-Cx43 antibody comprise a heavy chain having at least 80%, at least 85%, at least 90%, at least 95%, or at least 99% sequence identity to SEQ ID NO: 11; and a light chain having at least 80%, at least 85%, at least 90%, at least 95%, or at least 99% sequence identity to any one of SEQ ID NO: 10. In some other specific embodiments, the anti-Cx43 antibody comprise a heavy chain having at least 80%, at least 85%, at least 90%, at least 95%, or at least 99% sequence identity to SEQ ID NO: 12; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 13; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 14; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 15; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 16; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 17; and a light chain having at least 80%, at least 85%, at least 90%, at least 95%, or at least 99% sequence identity to any one of SEQ ID NO: 10. In some yet other specific embodiments, the anti-Cx43 antibody comprise a heavy chain having at least 80%, at least 85%, at least 90%, at least 95%, or at least 99% sequence identity to SEQ ID NO: 18; and a light chain having at least 80%, at least 85%, at least 90%, at least 95%, or at least 99% sequence identity to any one of SEQ ID NO: 10.

In some embodiments, the anti-Cx43 antibody comprises a heavy chain identical to any one of SEQ ID NOs: 9 and 11-18, and/or a light chain identical to any one of SEQ ID NO: 10. In a preferred embodiment, the anti-Cx43 antibody comprises a heavy chain identical to SEQ ID NO: 9, and a light chain identical to any one of SEQ ID NO: 10. In one embodiment, the anti-Cx43 antibody comprises a heavy chain identical to SEQ ID NO: 11, and a light chain identical to any one of SEQ ID NO: 10. In another embodiment, the anti-Cx43 antibody comprises a heavy chain identical to SEQ ID NO: 12, and a light chain identical to any one of SEQ ID NO: 10. In yet another embodiment, the anti-Cx43 antibody comprises a heavy chain identical to SEQ ID NO: 13, and a light chain identical to any one of SEQ ID NO: 10. In yet another embodiment, the anti-Cx43 antibody comprises a heavy chain identical to SEQ ID NO: 14, and a light chain identical to any one of SEQ ID NO: 10. In yet another embodiment, the anti-Cx43 antibody comprises a heavy chain identical to SEQ ID NO: 15, and a light chain identical to any one of SEQ ID NO: 10. In yet another embodiment, the anti-Cx43 antibody comprises a heavy chain identical to SEQ ID NO: 16, and a light chain identical to any one of SEQ ID NO: 10. In yet another embodiment, the anti-Cx43 antibody comprises a heavy chain identical to SEQ ID NO: 17, and a light chain identical to any one of SEQ ID NO: 10. In yet another embodiment, the anti-Cx43 antibody comprises a heavy chain identical to SEQ ID NO: 18, and a light chain identical to any one of SEQ ID NO: 10.

In various embodiments, provided herein is an antibody that binds an epitope located within, partially or entirely, the amino acid sequence of FLSRPTEKTI (SEQ ID NO: 19). In some embodiments, the epitope can comprise one or more amino acids selected from the group consisting of F1, S3, R4, P5, T6, E7, K8, T9, or 110 of SEQ ID NO: 19. In one embodiment the epitope consists of F1, S3, R4, P5, T6, E7, K8, T9 and 110 of SEQ ID NO: 19. In some embodiments, the epitope can include all ten amino acids of SEQ ID NO: 19. In certain embodiments, the epitope consists of all ten amino acids of SEQ ID NO: 19.

The anti-Cx43 antibody is substantially pure and desirably substantially homogeneous (i.e., free from contaminating proteins, etc.). “Substantially pure” antibody means a composition comprising at least about 90% antibody by weight, based on total weight of the protein in the composition, at least about 95% or 97% by weight. “Substantially homogeneous” antibody means a composition comprising protein wherein at least about 99% by weight of protein is specific antibody, e.g., anti-Cx43 antibody, based on total weight of the protein.

In some embodiments, the anti-Cx43 antibody is a humanized antibody. In some embodiments, the anti-Cx43 antibody is a monoclonal antibody. In some embodiments, the anti-Cx43 antibody is a humanized monoclonal antibody.

### Pharmaceutical Formulation Comprising Anti-Cx43 Antibody

One aspect of the present disclosure provides a pharmaceutical formulation for treating osteoarthritis in a subject comprising an anti-Cx43 antibody. In some embodiments, the anti-Cx43 antibody blocks or inhibits the opening of Cx43 hemichannels in osteocytes. The anti-Cx43 antibody can be a full antibody or an antigen-binding fragment thereof.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent may be coated m a material to protect the compm.md from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, or by the use of surfactants, such as polysorbate, sodium dodecyl sulfate, and nonionic surfactant. In many cases, it may be useful to include isotonic agents, for example, sugars, polyalcohols such as mannitol, sorbitol, or sodium chloride in the composition. Prolonged absorption of the injectable compositions can he brought about by including in the composition, an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration. Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol phenol sorbic acid, and the like. it may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions described herein can also have various viscosities or osmolarities. Methods of measuring viscosity of antibody formulations are known to those in the art, and can include, e.g., a rheometer (e.g., Anton Paar MCR301 Rheometer with either a 50 mm, 40 mm or 20 mm plate accessory). Methods of measuring osmolarity of antibody formulations are known to those in the art, and can include, e.g., an osmometer (e.g., an Advanced Instrument Inc 2020 freezing point depression osmometer).

The pharmaceutical compositions described herein can also have various pH levels. The pH of the pharmaceutical composition can be adjusted by any method known in the art, such as, for example, addition of a buffer.

In some embodiments, the pharmaceutical composition includes an anti-Cx43 antibody described herein, a histidine/histidine hydrochloride buffer, Polysorbate 80, and sucrose. In some embodiments, the pharmaceutical composition includes about 40 mg/mL to about 60 mg/mL of an anti-Cx43 antibody described herein; about 10 mM to about 40 mM histidine/histidine hydrochloride buffer; about 0.005% w/v to about 0.05% w/v Polysorbate 80; and about 1% w/v to about 20% w/v sucrose. In some embodiments, the pharmaceutical composition includes about 50 mg/mL of an anti-Cx43 antibody described herein; about 20 mM histidine/aspartic acid buffer; about 0.02% w/v Polysorbate 80; and about 8% w/v sucrose. In some embodiments, the pharmaceutical composition has a pH of between about 5.4 to about 5.6. In some embodiments, the pharmaceutical composition has a pH of about 5.5.

It may be advantageous to formulate parenteral compositions or unit dosage form for ease of administration and uniformity of dosage. Unit dosage form as used herein refers to physically discrete units suited as unitary dosages for the patients to be treated: each unit contains a predetermined quantity of active agent calculated to produce the desired therapeutic effect in association with any required pharmaceutical carrier. The specification for unit dosage forms is dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitations, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous injection and infusion are often (but not exclusively) used for antibody administration.

The formulations described herein are administered to a subject in need of treatment with the anti-Cx43 antibodies, preferably a human, in accord with known methods, such as intravenous administration as a bolus or by continuous infusion over a period of time, by intramuscular, intradermal, intraperitoneal, intracerobrospinal, subcutaneous, intra-articular, intrasynovial, intrathecal, oral, topical, or inhalation routes.

In some embodiments, the anti-Cx43 antibodies are administered to a subject by intravenous or subcutaneous (i.e., beneath the skin) administration. For such purposes, the formulation may be injected using a syringe. However, other devices for administration of the formulation are available such as injection devices (e.g., the INJECT-EASE™ and GENJECT™ devices); injector pens (such as the GENPEN™); auto-injector devices, needleless devices (e.g., MEDIJECTOR™ and BIOJECTOR™); and subcutaneous patch delivery systems.

In a specific embodiment, the present disclosure is directed to kits for a single dose-administration unit. Such kits comprise a container of an aqueous formulation of therapeutic protein or antibody, including both single or multi-chambered pre-filled syringes. Exemplary pre-filled syringes are available from Vetter GmbH, Ravensburg, Germany.

### III. Method of Treating Osteoarthritis

Certain aspects of the present disclosure provide methods of treating osteoarthritis in a subject. The methods can comprise blocking the opening of Cx43 hemichannels in osteocytes in a subject in need thereof by using, e.g., an anti-Cx43 antibody. The Cx43 modulation can be a stand-alone therapy for osteoarthritis, or in conjunction with other therapies. The osteoarthritis can affect any joint in a subject, such as the hands and fingers, wrists, knees, hips, feet, neck and back.

Osteoarthritis is a prevalent type of arthritis that causes the degeneration of joints, including articular cartilage and subchondral bone. The pathology of osteoarthritis is characterized by a loss of articular cartilage leading to narrowing of joint space, increased joint friction and potential structure remodeling. Current treatment options of osteoarthritis include exercise, lifestyle changes, analgesics, and, in the case of severe symptoms, joint replacement surgery. However, there is a lack of specific pharmaceutical interventions for the treatment of osteoarthritis.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells, and they mediate intercellular communication. Hemichannels are a separate entity from gap junction channels. Hemichannels permit the exchange of molecules between the intracellular compartments and the extracellular environment.

Chondrocytes express hemichannels known as connexin (Cx) 43 hemichannels. Under normal conditions, Cx43 hemichannels in chondrocytes remain closed. Inflammatory conditions and mechano-stimulation can induce opening of the Cx43 hemichannels, leading to the release of pro-inflammatory factors that contribute to the inflammation associated with osteoarthritis. Inhibiting the opening of Cx43 hemichannels in chondrocytes (e.g., using an anti-Cx43 antibody) can thus suppress the development and progression of ostearthritis in a subject.

Cx43 is also known as gap junction alpha-1 protein (GJA1), which is a 43.0 kDa protein composed of 382 amino acids (NCBI Reference Sequence: NP 000156.1). GJA1 contains a long C-terminal tail, an N-terminal domain, and multiple transmembrane domains. The protein passes through the phospholipid bilayer four times, leaving its C- and N-terminals exposed to the cytoplasm. The C-terminal tail is composed of 50 amino acids and includes post-translational modification sites, as well as binding sites for transcription factors, cytoskeleton elements, and other proteins. As a result, the C-terminal tail is central to functions such as regulating pH gating and channel assembly. Notably, the DNA region of the GJA1 gene (NCBI Gene ID: 2697) encoding this tail is highly conserved, indicating that it is either resistant to mutations or becomes lethal when mutated. Meanwhile, the N terminal domain is involved in channel gating and oligomerization and, thus, may control the switch between the channel's open and closed states. The transmembrane domains form the gap junction channel while the extracellular loops facilitate proper channel docking. Moreover, two extracellular loops form disulfide bonds that interact with two hexamers to form a complete gap junction channel.

“Treatment” refers to therapeutic treatment. Those in need of treatment include those already with disease. Hence, the subject, e.g., a human, to be treated herein may have been diagnosed as suffering from a disease, such as osteoarthritis. A disease, e.g., osteoarthritis, is “inhibited” or “treated” if at least one symptom (as determined by responsiveness/non-responsiveness, or indicators known in the art and described herein) of the condition is alleviated, terminated, slowed, minimized, or prevented. The terms “patient” and “subject” are used interchangeably herein.

The term “subject” or “patient” refers to either a human or non-human, such as primates, mammals, and vertebrates. In particular embodiments, the subject is a human.

Treatment can be suitably administered to subjects, particularly humans, suffering from, having, susceptible to, or at risk of developing osteoarthritis. Determination of those subjects “at risk” can be made by any objective or subjective determination by a diagnostic test or opinion of a subject or health care provider. Identifying a subject in need of such treatment can be in the judgment of a subject or a health care professional and can be subjective (e.g., opinion) or objective (e.g., measurable by a test or diagnostic method).

The anti-Cx43 antibody for use in any of the methods disclosed herein can be stored as a lyophilized solid or an aqueous formulation, or any other forms known in the art. In the case of an anti-Cx43 antibody which is stored as a lyophilized solid, the antibody is reconstituted in a solution such as water (e.g., for injection) prior to administration. If prepared for infusion either from a lyophilized form or an aqueous formulation, the final concentration, e.g., after dilution of the reconstituted antibody (e.g., in a saline, Ringer's or 5% dextrose infusion system) of the anti-Cx43 antibody can be about 0.1 mg/ml to about 80 mg/ml for administration. The final concentration may be about 0.1 mg/ml to about 80 mg/ml, about 0.5 mg/ml to about 70 mg/ml, about 1 mg/ml to about 60 mg/ml, about 5 mg/ml to about 50 mg/ml, about 10 mg/ml to about 40 mg/ml, about 15 mg/ml to about 30 mg/ml, or about 20 mg/ml to about 25 mg/ml. In some embodiments, the final dosage form may be at a concentration of about 0.1 mg/ml, about 0.5 mg/ml, about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml or higher than 80 mg/ml.

The term “effective amount,” as used herein, refers to that amount of an agent, such as an anti-Cx43 antibody, which is sufficient to effect treatment, prognosis or diagnosis of osteoarthritis, when administered to a patient or a subject. Dosage regimens may be adjusted to provide the optimum therapeutic response. An effective amount is also one in which any toxic or detrimental effects (side effects) of the agent are minimized and/or outweighed by the beneficial effects. A therapeutically effective amount will vary depending upon the patient and disease condition being treated, the weight and age of the patient, the severity of the disease condition, the manner of administration, course of the condition, patient's clinical history and response to anti-Cx43 antibody and the like, which can readily be determined by one of ordinary skill in the art. For example, an effective amount or a dose of the anti-Cx43 antibody ranges from about 0.01 mg/kg to about 1000 mg/kg. In some embodiments, the effective amount or the dose of the anti-Cx43 antibody is about 0.01 mg/kg to about 900 mg/kg, about 0.1 mg/kg to about 800 mg/kg, about 0.5 mg/kg to about 700 mg/kg, about 1 mg/kg to about 600 mg/kg, about 1.5 mg/kg to about 500 mg/kg, about 2 mg/kg to about 400 mg/kg, about 5 mg/kg to about 300 mg/kg, about 10 mg/kg to about 200 mg/kg, about 15 mg/kg to about 100 mg/kg, about 20 mg/kg to about 50 mg/kg, about 25 mg/kg to about 45 mg/kg, or about 30 mg/kg to about 40 mg/kg. In some embodiments, the effective amount or the dose of the anti-Cx43 antibody is about 0.01 mg/kg, about 0.1 mg/kg, about 1 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, or more than 1000 mg/kg.

In specific embodiments, an effective amount or a dose of the anti-Cx43 antibody is about 0.01 mg/kg to about 100 mg/kg. In some embodiments, the effective amount or a dose of the anti-Cx43 antibody is 15 mg/kg. In some embodiments, the effective amount or a dose of the anti-Cx43 antibody is 25 mg/kg. In some embodiments, the effective amount or a dose of the anti-Cx43 antibody is 50 mg/kg.

In some embodiments, an effective amount or a dose of the anti-Cx43 antibody can range from about 1 mg to about 800 mg. In some embodiments, the effective amount or the dose of the anti-Cx43 antibody is about 2 mg to 7000 mg, about 5 mg to 6000 mg, about 10 mg to 5000 mg, about 15 mg to 4000 mg, about 20 mg to 3000 mg, about 25 mg to 2000 mg, about 30 mg to 1000 mg, about 40 mg to 900 mg, about 50 mg to 800 mg, about 60 mg to 700 mg, about 70 mg to 600 mg, about 80 mg to 500 mg, about 90 mg to 400 mg, about 100 mg to 300 mg, or about 150 mg to 250 mg. In some embodiments, the effective amount or the dose of the anti-Cx43 antibody is less than 1 mg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1250 mg, about 1500 mg, about 1750 mg, about 2000 mg, about 2250 mg, about 2500 mg, about 2750 mg, about 3000 mg, about 4000 mg, about 5000 mg, about 6000 mg, about 7000 mg, about 8000 mg, or more than 8000 mg.

In the methods as described herein, an effective amount or at least one dose of the anti-Cx43 antibody is administered about once every day, about once every 2 days, about once every 3 days, about once every 4 days, about once every 5 days, about once every 6 days, about once every week, about once every 8 days, about once every 9 days, about once every 10 days, about once every 11 days, about once every 12 days, about once every 13 days, about once every 2 weeks, about once every 15 days, about once every 16 days, about once every 17 days, about once every 18 days, about once every 19 days, about once every 20 days, about once every 3 weeks, about once every 22 days, about once every 23 days, about once every 24 days, about once every 25 days, about once every 26 days, about once every 27 days, about once every 4 weeks, about once every 29 days, about once every 30 days, about once every 31 days, about once every 32 days, about once every 33 days, about once every 34 days, about once every 5 weeks, about once every 36 days, about once every 37 days, about once every 38 days, about once every 39 days, about once every 40 days, or about once every 41 days, about once every 6 weeks, about once every 7 weeks, about once every 8 weeks, about once every 9 weeks, about once every 10 weeks, about once every 11 weeks, about once every 12 weeks, about once every 13 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks or 6 months, or about once every more than 24 weeks or 6 months.

In some specific embodiments, an effective amount or a dose of the anti-Cx43 antibody is administered about once every week. In one particular embodiment, a dose of the anti-Cx43 antibody is administered once every week. In another particular embodiment, a dose of the anti-Cx43 antibody is administered once every week for 1, 2, 3, 4, 5, 6, 7, 8, 9, 10, or longer than 10 weeks.

According to certain embodiments of the present disclosure, multiple doses of an anti-Cx43 antibody (or a pharmaceutical composition comprising a combination of an anti-Cx43 antibody and any of the additional therapeutically active agents mentioned herein) may be administered to a subject over a defined time course. The methods according to this aspect of the disclosure comprises sequentially administering multiple doses of an anti-Cx43 antibody of the disclosure to a subject.

As used herein, “sequentially administering” means that each dose of anti-Cx43 antibody is administered to the subject at a different point in time, e.g., on different days separated by a predetermined interval (e.g., hours, days, weeks or months). The present disclosure includes methods which comprise sequentially administering to the patient a single initial dose of an anti-Cx43 antibody, followed by a second dose of the anti-Cx43 antibody, followed by a third dose of the anti-Cx43 antibody, followed by a fourth dose of the anti-Cx43 antibody, and optionally followed by one or more subsequent doses of the anti-Cx43 antibody. The anti-Cx43 antibody may be administered at a dose of between 0.01 mg/kg to about 1000 mg/kg.

The terms “first dose,” “second dose,” “third dose,” and “fourth dose” refer to the temporal sequence of administration of the anti-Cx43 antibody of the disclosure. Thus, the “first dose” is the dose which is administered at the beginning of the treatment regimen (also referred to as the “baseline dose”); the “second doses” are the doses which are administered after the first dose; the “third doses” are the doses which are administered after the second doses; and the “fourth doses” are the doses which are administered after the third doses. The first, second, third and fourth doses may all contain the same amount of anti-Cx43 antibody, but generally may differ from one another in terms of frequency of administration. In certain embodiments, however, the amount of anti-Cx43 antibody contained in the first, second, third, fourth and/or doses following the fourth dose varies from one another (e.g., adjusted up or down as appropriate) during the course of treatment.

In some embodiments of the methods of the disclosure, the method comprises administering a first dose and a second dose of the anti-Cx43 antibody. In some embodiments, the first dose of the anti-Cx43 antibody is administered at day 1 of treatment. In some embodiments, the second dose of the anti-Cx43 antibody is administered at day 8 of treatment. In some embodiments, the methods disclosed herein comprise administering a third dose of the anti-Cx43 antibody. In some embodiments, the third dose of the anti-Cx43 antibody is administered on day 15 of treatment. In some embodiments, the method further comprises administering a fourth dose of the anti-Cx43 antibody. In some embodiments, the fourth dose of the anti-Cx43 antibody is administered on day 22 of treatment. In some embodiments, the method further comprises administering subsequent doses of the anti-Cx43 antibody after the fourth dose. In some embodiments, the subsequent doses of the anti-Cx43 antibody are administered weekly after the fourth dose.

In some embodiments of the methods of the disclosure, the method comprises administering an effective amount of an anti-Cx43 antibody according to the following dose regimens: a first dose on day 1 of treatment, a second dose on day 8 of treatment, a third dose on day 15 of treatment, and a fourth dose on day 22 of treatment. In some embodiments, the method further comprises administering subsequently doses of the anti-Cx43 antibody weekly after the fourth dose.

The effective amount or at least one dose of the anti-Cx43 antibody is suitably administered to the patient at one time or over a series of treatments and may be administered to the patient at any time from diagnosis onwards. The anti-Cx43 antibody may be administered as the sole treatment or in conjunction with other drugs or therapies useful in treating osteoarthritis.

In the methods as described herein, an effective amount or a dose of the anti-Cx43 antibody can be administered to a patient over less than 5 minutes, about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more than 180 minutes. In some specific embodiments, the effective amount or the dose of the anti-Cx43 antibody can be administered to a patient over about 30 minutes. In one specific embodiment, the effective amount or the dose of the anti-Cx43 antibody can be intravenously administered to a patient over 30 minutes.

In the methods described herein, an anti-Cx43 antibody is administered to a patient. If the anti-Cx43 antibody is in a formulation which is in a solid, e.g., dry state, the process of administration can comprise a step of converting the formulation to a liquid state. In one aspect, a dry formulation can be reconstituted, e.g., by a liquid as described above, for use in injection, e.g. intravenous, intradermal, intramuscular, intraperitoneal or subcutaneous injection. In another aspect, a solid or dry formulation can be administered topically, e.g., in a patch, cream, aerosol or suppository.

In the methods described herein, the anti-Cx43 antibody may be administered to a subject alone or in conjunction with another therapy. The anti-Cx43 antibody can be administered before, along with or subsequent to administration of the additional therapy. In one embodiment, the dose of the co-administered therapy can be decreased over time or completely tapered during the period of treatment by the anti-Cx43 antibody. In some embodiments, the additional comprises one or more of physical therapy, adaptive aids, a steroid, surgery (e.g., joint replacement surgery), cell-based therapy, or a small molecule drug.

In one aspect, the improved effectiveness of a combination according to the disclosure can be demonstrated by achieving therapeutic synergy. The term “therapeutic synergy” or “synergistic effect” is used when the combination of two products at given doses is more efficacious than the best of each of the two products alone at the same doses. In one example, therapeutic synergy can be evaluated by comparing a combination to the best single agent using estimates obtained from a two-way analysis of variance with repeated measurements (e.g., time factor) on an osteoarthritis severity parameter

The patient or subject, before, during, or after being treated with the anti-Cx43 antibody, is examined for osteoarthritis related parameters using technologies and methods known in the art. Non-limiting examples of the common medical technologies and methods used to examine and diagnose osteoarthritis include: physical examination by a physician, physical performance tests, blood tests, X-rays, magnetic resonance imaging (MRI) radiography, computerized tomography (CT) scanning the Western Ontario and McMaster Universities Arthritis (WOMAC) Index, the Knee Injury and Osteoarthritis Outcome Score (KOOS) questionnaire, quality of life (QOL) assessment (e.g., using the SF-12 Scale or the Assessment of Quality of Life-6-Dimension or “AQoL-6D” method), Patient Global Impression of Change (PGIC) questionnaire, maximum voluntary isometric strength measurement (e.g., using a dynatometer), dynamic weight bearing (DMB) testing, the Observational Gait Score (OGS), numeric rating scale for assessing pain degree. The choice of technologies and methods, and the frequency of examination can be determined and/or adjusted by a person skilled in the art based on the patient's or subject's specific conditions.

In some embodiments, the subject, before, during, or after being treated with the anti-Cx43 antibody, is examined for improvement of the osteoarthritis as assessed by at least one indicator of osteoarthritis severity. In some embodiments, at least one indicator of osteoarthritis severity is assessed at least one day to one week after the effective amount or a dose of the anti-Cx43 antibody is administered to the subject. In some embodiments, at least one indicator of osteoarthritis severity is assessed a day, two days, three days, four days, five days, six days, or a week after the effective amount or the dose of anti-Cx43 antibody is administered to the subject. In some embodiments, at least one indicator of the osteoarthritis severity comprises pain threshold of the arthritis, gait score, and/or weight bearing difference.

In some embodiments, treatment with the anti-Cx43 antibody for osteoarthritis improves the disease symptoms as assessed by pain threshold of the arthritis, gait score, and/or weight bearing difference comparing with a control, which can include but is not limited to an osteoarthritis population who receive or failed the same lines of therapy as the subject, but do not receive the anti-Cx43 antibody for osteoarthritis.

In some embodiments, the subject treated with the anti-Cx43 antibody for osteoarthritis has a duration of response of at least 5 days, at least 10 days, at least 15 days, at least 20 days, at least 25 days, at least 1 month, at least 2 months, at least 3 months, or more.

In some embodiments, the subject treated with the anti-Cx43 antibody for osteoarthritis has a time to response less than 3 months, less than 2 months, less than 1 months, less than 25 days, less than 20 days, less than 15 days, less than 10 days, less than 7 days, less than 6 days, less than 5 days, less than 4 days, less than 3 days, less than 2 days, or less than 1 day.

As used herein, the “pain threshold” refers to the lowest intensity at which a given stimulus is perceived as painful, and it is relatively constant across individuals for a given stimulus. Injury or disease (e.g., osteoarthritis) may result in a decrease in the pain threshold of the subject at a particular location (e.g., a limb). The pain threshold can be a mechanical pain threshold, or a pressure pain threshold, and may be measured locally (e.g., on an affected joint) or remotely (e.g., on the neighboring muscle) by any suitable method known in the art.

In some embodiments, treatment of a subject having osteoarthritis with at least one dose of the anti-Cx43 antibody improves the pain threshold of the arthritis in the subject. In some embodiments, treatment of a subject having the osteoarthritis with the at least one dose of the anti-Cx43 antibody increases the pain threshold of the arthritis by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more.

As used herein, “gait” refers to the way limbs move during locomotion. The gait of a subject may be affected presence of a disease or disorder that affects locomotion (e.g., osteoarthritis). A “gait score” refers to any scale or scoring system known in the art for evaluating and analyzing the gait of a subject. Depending on the gait scoring system used, an improvement in the gait of a subject may be reflected as an increase or a decrease in the gait score of said subject.

In some embodiments, treatment of a subject having osteoarthritis with the anti-Cx43 antibody improves the gait score of the subject. In some embodiments, treatment of a subject having the osteoarthritis with the at least one dose of the anti-Cx43 antibody improves the gait score of the subject by at 0.1, at least 0.2, at least 0.3, at least 0.4, at least 0.5, at least 0.6, at least 0.7, at least 0.8, at least 0.9, at least 1, at least 1.2, at least 1.5, at least 1.8, at least 2, at least 2.2, at least 2.5, at least 3, at least 3.5, or more. In some embodiments, treatment of a subject having the osteoarthritis with the at least one dose of the anti-Cx43 antibody improves the gait score of the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more.

As used herein, the “weight bearing” refers to the amount of weight or force a specific limb is able to hold. The weight bearing may be determined by any method known in the art. A reduction in weight bearing can be observed in the case injury or disease (e.g., osteoarthritis), and/or may be recommended by a physician to reduce risk of injury or pain to the subject. The “weight bearing difference” as used herein refers to the difference between the weight bearing of a limb during injury or disease (e.g., osteoarthritis) state as compared to the weight bearing of a corresponding limb without the injury or disease. A decrease in the weight bearing difference indicates that the weight bearing of the limb is closer to its full weight bearing.

In some embodiments, treatment of a subject having osteoarthritis with the anti-Cx43 antibody improves the weight bearing difference of the subject. In some embodiments, treatment of a subject having the osteoarthritis with the at least one dose of the anti-Cx43 antibody decreases the weight bearing difference of the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more.

As used herein, a “response” or “being responsive” to a treatment refers to the subject having improvement of at least one parameter of disease progression. The subject can have partial response or complete response to a treatment. The response to a treatment can be determined based on methods known in the art. A person skilled in the art can determine the proper methods based on the type of diseases being evaluated. Non-limiting examples of the methods include pain threshold of the arthritis, gait score, and weight bearing difference, and other methods described herein.

### EXAMPLES

The following examples are included to demonstrate preferred embodiments of the invention. It should be appreciated by those of skill in the art that the techniques disclosed in the examples which follow-represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Effects of Treatment with an Anti-Cx43 Antibody on a Rat Model of Osteoarthritis

This Example describes the evaluation of the effects of treatment with an anti-Cx43 in a rat model of osteoarthritis.

### Study Objective

The purpose of the study was to test the effect of the anti-Cx43 antibody ALMB-0166 on osteoarthritis symptoms in a rat model of osteoarthritis caused by partial meniscectomy and anterior cruciate ligament resection (pMMx+ACLT) when administered by intravenous injection once a week for four weeks.

### Regulatory Compliance

The experiment complied with Standard Operating Procedures (SOPs).

### Materials and Methods

### Test Materials

The anti-Cx43 antibody used in the study was ALMB-0166 (AlaMab Therapeutics Inc. Duloxetine hydrochloride (MedChemExpress) was used as a positive control. Other materials used in the study included isoflurane (RWD Life Science, Inc.), phosphate buffered saline (Thermo), and normal saline (Anhui Shuanghe Pharmaceutical Co., Ltd).

For animal dosing, a 0.5 mg/mL of duloxetine hydrochloride by dissolving the drug in normal saline, while two ALMB-0166 solutions, one at 3 mg/ml and another at 5 mg/mL were prepared by diluting the antibody stock solution with PBS.

### Equipment

Equipment used in this study included a non-rebreathing anesthesia machine (Midmark Corporation, model VWR), Von Frey filaments (IITC, Life Science, USA), and a Linton incapacitance tester (UK).

### Animals and Husbandry

Male Sprague Dawley (SD) rats (Specific Pathogen Free grade; Shanghai Slac Laboratory Animal Co. Ltd) were obtained in the study. Each rat was weight at the start of treatment, with the average weight ranging from 200-220 grams. Upon arrival at the test facility, the animals were housed at five rats per cage and acclimated for five to seven days prior to initiation of treatment.

Animals were kept in an environment set to maintain a temperature of 23±2° C., with humidity of 40-70%, and a 12-hour light/12-hour dark cycle. The 12-hour dark cycle was temporarily interrupted to accommodate study procedures. SPF Rat Growth Breeding Feed (Beijing Keao Xieli Feed Co., Ltd)) was provided ad libitum throughout the in-life part of the study. Reverse Osmosis water was available ad libitum. Animals were free to access both food and water during the whole course of study.

The animals used in the study were selected based on overall health and acclimation to caging, and a total of 48 rats were selected for participation in the study.

### Study Design

### Animal Groups and Drugs

For the study, animal groups were formed as indicated in Table 1 below. The administration of the respective treatments was started a week after partial medial meniscectomy in combination with anterior cruciate ligament transection (pMMx+ACLT) of the rats.

TABLE 1
Treatment and dosing information for animal test groups.

| | | | Dosing | | |
|---|---|---|---|---|---|
| | | Animal | Volume | Frequency of | Route of |
| Group | Treatment | Number | (mg/kg) | Administration | Administration |
| 1 | Control (no osteoarthritis | 8 | — | — | — |
| | induction) | | | | |
| 2 | Model (vehicle-treated) | 8 | — | Once/week, for 4 | Intravenous (i.v.) |
| | | | | weeks | |
| 3 | Duloxetine 5 mg/kg | 8 | 5 | Daily, for 4 weeks | Intragastric (i.g.) |
| 4 | ALMB-0166 15 mg/kg | 8 | 15 | Once/week, for 4 | Intravenous (i.v.) |
| | | | | weeks | |
| 5 | ALMB-0166 25 mg/kg | 8 | 25 | Once/week, for 4 | Intravenous (i.v.) |
| | | | | weeks | |
| 6 | ALMB-0166 50 mg/kg | 8 | 50 | Once/week, for 4 | Intravenous (i.v.) |
| | | | | weeks | |

### Models and Methods

After acclimation of the animals, the animals were evaluated for normal pain threshold (on Day 1 of study) using the Von Frey, or Up-Down, method, and for biped balance degree of the knee joint on the surgical side (right hind limb) using a biped balance tester. The rats were then anesthetized with isoflurane, and the knee joints were depilated and disinfected. Osteoarthritis was induced in the animals by partial medial meniscectomy in combination with anterior cruciate ligament transection (pMMx+ACLT). Briefly, the skin and joint cavity close to the inner side of the right knee were cut, the knee joint was placed in the maximum flexion state, with the joint cavity exposed and the medial meniscus separated. The medial ⅓ meniscus and the anterior cruciate ligament were then cut-off. For the control animal group (no osteoarthritis induction), only the skin was cut to open the joint cavity without removing the ligament or meniscus. The date of the osteoarthritis model establishment was Day 8 of the study.

One week after model establishment (Day 15 of study), before administration of treatment, the basic pain threshold of plantar pain was evaluated on the operation side for each of the animals, and the osteoarthritis model rats were evenly divided according to the basic Von Frey reaction value (2-5), with 8 animals used for each of the osteoarthritis model group (vehicle-treatment), the positive control drug (duloxetine) 5 mg/kg group, and the ALMB-0166 15 mg/kg, 25 mg/kg, and 50 mg/kg dose groups. After grouping, drug treatment was started on Day 15 of study. For each of the ALMB-0166-treated test groups, the treatment was administered intravenously, once a week, with four total doses (on Day 15, 22, 29, and 36 of the study, corresponding to day 1, 8, 15 and 22 of treatment). For the positive control group, duloxetine was administered orally every day until the end of the experimental cycle.

Weight measurements were taken for animals in each test group on Days 15, 22, 29 and 36 of the study. On Days 16, 23, 30 and 37, the gait of each animal, the change of plantar pain threshold induced by knee joint lesions on the operative side, and the weight bearing of both lower limbs were measured for each animal was scored on according to the Coderre scoring method, the Up-Down method, and bipedal balance pain testing, respectively.

### Coderre Scoring Method

Coderre gait method was used to measure the gait of each animal. The gait was measured according to the following scoring: standard (Grade 0: normal walking, 0 point); Level 1 (sight claudication, slight bending below knee joint, 2 points); Level 2 (claudication, the affected limb can touch the ground, 4 points); Level 3 (severe claudication, the affected limb leaves the ground and walk on three feet, 6 points).

### Mechanical Pain Threshold Measurement

The Up-Down method was used to measure the plantar pain threshold. Following the Up-Down methos, each rat was placed in a transparent enclosure with a sieve-shaped metal plate at the bottom, and the animal was allowed to adapt to about 15 minutes until the autonomous exploration and grooming activities stop. Von Frey probes were then used to vertically stimulate the middle of the hind plantar of each rat to make the plantar slightly s-shaped for 5 second, and the paw withdrawal response was observed. A positive response was noted if the paw was sharply withdrawn or there was flinching upon removal. The interval between each measurement was about 10 sec. If no response was observed after probe stimulation, it was regarded as a negative reaction.

For scoring the mechanical pain, the rats were stimulated with a series of Von Frey probes of 0.4, 0.6, 1, 1.4, 2, 4, 6, 8, 10, and 15 g). Testing was start with a 2 g probe, and if a positive reaction occurred, it was recorded as an “X,” and testing was continued with a 1.4 g probe. If no positive reaction was observed with the 2 g probe, it was recorded as an “O,” and testing was continued with a 4 g probe. Testing was continued accordingly, using the probes successively without circumvention. Each response was recorded, and when a “OX” or “XO” appeared for the first time, it was tested four times to obtain a series of sequences, such as “OXOXOX.” If the rat did not have a positive reaction after using a 15 g probe, the 50% withdrawal threshold (50% MWT) was recorded as 15 g. If a positive reaction was induced even by the 0.4 g probe, the 50% MWT was recorded as 0.4 g. If 15 g and 0.4 g probes are not used, calculate according to the following formula: 50% MWT (g)=(10 [Xf+kδ])/10000, with “50% MWT” defined as a positive reaction in 50% chance under that stimulus intensity, “Xf” as the value of the last used Von Frey probe (in log units), “K” as the value that can be obtained by looking up the table according to the measured sequence, and “8” as the average value of the difference between the series of stimuli (in logarithmic units, defined as 0.224).

### Biped Balance Measurement

A bipedal balance pain tester was used to evaluate the weight bearing of both lower limbs for each animal. Each animal was placed in the plexiglass cover of the biped balance pain instrument. After the animal completed their exploratory behavior, the forelimbs of the rats laid on the incline of the device, and the hind limbs stood on the left and right balance plates respectively. Once the animals were inactive, the average weight bearing capacity of the left and right hind limbs were measured within 5 seconds from the inactivity. The measurements were repeated three times for each animal, with an interval of 10 seconds between each replicate measurement.

### Statistical Analysis

The gait scoring, plantar pain threshold, and weight bearing differential were expressed as the mean and standard error (Mean±SD). The data between model group and administration group at multiple time points were analyzed by repeated measures analysis of variance method with SPSS Statistics 21 software, and Tamhane's method for subsequent analysis. P<0.05 was considered to have a significant difference, and p<0.01 was considered to have a very significant difference. The final data were plotted with GraphPad Prism 6 software.

### Results

General Clinical Symptoms of Rats after Administration

All animals had no visible abnormalities during the experimental period.

Effect of Treatment with Anti-Cx43 Antibody on Weight of Rats with Osteoarthritis Induced by pMMx+ACLT

The body weight of animals in each test group increased over time upon administration of the respective treatments (Table 2). No significant difference was observed between the groups.

TABLE 2
Weight of animal test groups over study period (Mean ± SEM, n = 8).

| | Weight (g) | | | | | |
|---|---|---|---|---|---|---|
| Group | Day 1 | Day 8 | Day 15 | Day 22 | Day 29 | Day 36 |
| Control (no | 225.0 ± 6.8 | 283.8 ± 12.0 | 334.5 ± 20.1 | 383.9 ± 26.8 | 423.1 ± 29.7 | 455.3 ± 30.5 |
| osteoarthritis | | | | | | |
| induction) | | | | | | |
| Model | 221.6 ± 5.3 | 278.6 ± 8.0 | 315.1 ± 12.0 | 362.9 ± 14.3 | 393.5 ± 20.4 | 436.1 ± 17.8 |
| (vehicle- | | | | | | |
| treated) | | | | | | |
| Duloxetine 5 | 223.8 ± 7.8 | 280.1 ± 11.2 | 317.8 ± 20.4 | 353.9 ± 23.0 | 389.9 ± 26.2 | 423.0 ± 30.0 |
| mg/kg | | | | | | |
| ALMB-0166 | 228.8 ± 9.8 | 284.1 ± 11.7 | 312.8 ± 9.7 | 358.0 ± 10.7 | 389.5 ± 17.1 | 416.3 ± 18.6 |
| 15 mg/kg | | | | | | |
| ALMB-0166 | 225.9 ± 7.9 | 282.6 ± 11.4 | 313.3 ± 13.2 | 358.8 ± 16.6 | 391.6 ± 20.3 | 422.5 ± 19.9 |
| 25 mg/kg | | | | | | |
| ALMB-0166 | 225.8 ± 6.6 | 277.6 ± 11.4 | 312.9 ± 23.2 | 359.0 ± 27.5 | 389.8 ± 27.6 | 420.8 ± 31.7 |
| 50 mg/kg | | | | | | |

Effect of Treatment with Anti-Cx43 Antibody on Gait of Rats with Osteoarthritis Induced by pMMx+ACLT

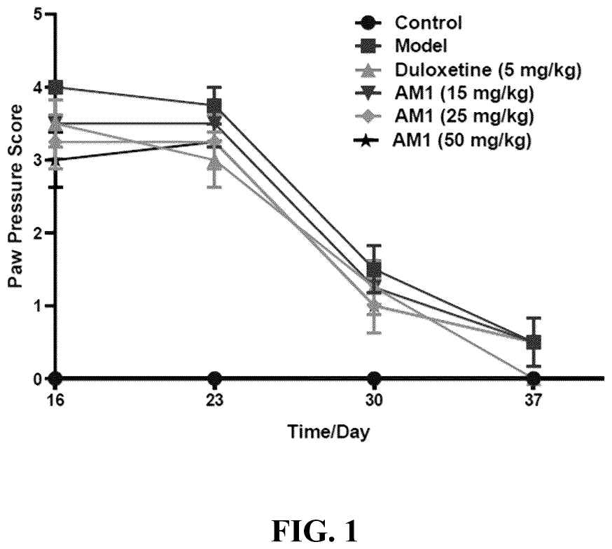

Osteoarthritis model rats exhibited lameness on Day 16 of the study period (after operation 1 week), with the gait score reaching an average of 3.8 between the test groups (Table 3 and FIG. 1). The gait score was alleviated over the course of treatment, with the gait score of the osteoarthritis model rats being comparable to control rats by Day 37 of the study (Table 3 and FIG. 1). A significant difference in gait score was observed in the osteoarthritis model rat test groups as compared to control (no osteoarthritis) rats (multivariate test, p<0.05, α=0.05; Table 3). While a decrease in gait score over time was observed for the ALMB-0166 and duloxetine dosage groups, no significant different was observed when compared to the vehicle-treated control group (multivariate test, p>0.05, α=0.05; Table 3).

TABLE 3
Gait score of animal test groups over study
period (Mean ± SEM, n = 8).

| | Gait Score | | | |
|---|---|---|---|---|
| Group | Day 16 | Day 23 | Day 30 | Day 37 |
| Control (no | 0.0 ± 0.0 | 0.0 ± 0.0 | 0.0 ± 0.0 | 0.0 ± 0.0 |
| osteoarthritis induction) | | | | |
| Model (vehicle-treated) | 4.0 ± 0.0^{#} | 3.8 ± 0.7^{#} | 1.5 ± 0.9^{#} | 0.5 ± 0.9 |
| Duloxetine 5 mg/kg | 3.5 ± 0.9 | 3.0 ± 1.1 | 1.3 ± 1.0 | 0.0 ± 0.0 |
| ALMB-0166 15 mg/kg | 3.5 ± 0.9 | 3.5 ± 0.9 | 1.3 ± 1.0 | 0.5 ± 0.9 |
| ALMB-0166 25 mg/kg | 3.3 ± 1.0 | 3.3 ± 1.0 | 1.0 ± 1.1 | 0.5 ± 0.9 |
| ALMB-0166 50 mg/kg | 3.0 ± 1.1 | 3.3 ± 1.0 | 1.0 ± 1.1 | 0.5 ± 0.9 |

Note:
^{#}= P < 0.01 vs. Control

Effect of Treatment with Anti-Cx43 Antibody on Mechanical Pain of Rats with Osteoarthritis Induced by pMMx+ACLT

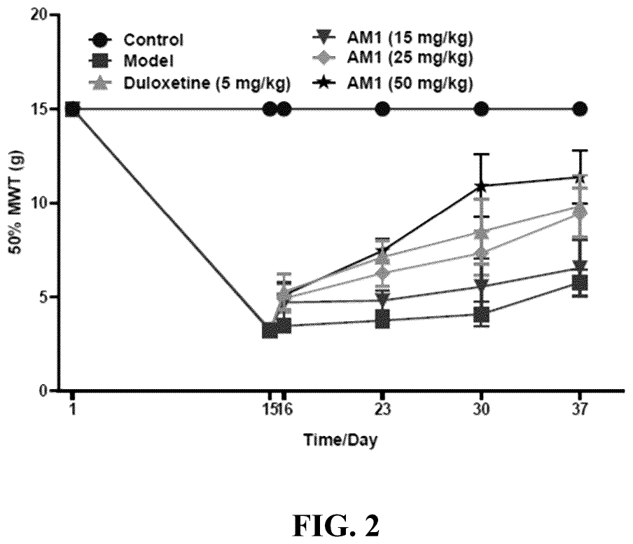

A baseline level of pain threshold of plantar pain (50% MWT) of about 3.5 g was observed in osteoarthritis model rats after a week of osteoarthritis induction. An increase in pain threshold of plantar pain (50% MWT) was observed over time after start administration of each treatment, as compared to Day 15 of study (Day 1 of treatment) (Tables 4-5, and FIG. 2). The improvement in pain threshold of plantar pain correlated with the number of doses of ALMB-0166 administered (Table 5, and FIG. 2).

TABLE 4
Pain threshold of plantar pain of animal test groups over study period (Mean ± SEM, n = 8).

| | 50% MWT (g) | | | | | |
|---|---|---|---|---|---|---|
| Group | Day 1 | Day 15 | Day 16 | Day 23 | Day 29 | Day 37 |
| Control (no | 15.00 ± 0.00 | 15.00 ± 0.00 | 15.00 ± 0.00 | 15.00 ± 0.00 | 15.00 ± 0.00 | 15.00 ± 0.00 |
| osteoarthritis | | | | | | |
| induction) | | | | | | |
| Model (vehicle- | 15.00 ± 0.00 | 3.21 ± 0.71^{#} | 3.44 ± 0.76^{#} | 3.74 ± 0.77^{#} | 4.07 ± 1.86^{#} | 5.76 ± 2.02^{#} |
| treated) | | | | | | |
| Duloxetine 5 mg/kg | 15.00 ± 0.00 | 3.23 ± 0.64 | 5.27 ± 2.72 | 7.11 ± 2.45** | 8.47 ± 4.92* | 9.84 ± 4.65* |
| ALMB-0166 15 | 15.00 ± 0.00 | 3.25 ± 0.69 | 4.70 ± 2.96 | 4.80 ± 1.53 | 5.53 ± 4.26 | 6.53 ± 4.30 |
| mg/kg | | | | | | |
| ALMB-0166 25 | 15.00 ± 0.00 | 3.26 ± 0.69 | 4.91 ± 2.17 | 6.26 ± 1.93** | 7.32 ± 3.35* | 9.43 ± 3.83* |
| mg/kg | | | | | | |
| ALMB-0166 50 | 15.00 ± 0.00 | 3.18 ± 0.76 | 5.09 ± 2.05^{##} | 7.45 ± 1.83** | 10.91 ± 4.76** | 11.38 ± 4.05** |
| mg/kg | | | | | | |

Note:
^{#}p < 0.01 vs. Control;
^{##}p = 0.52 vs. Control;
*p < 0.05,
**p < 0.01 vs. Model

TABLE 5
Increase rate of pain threshold of plantar pain of animal test
groups over study period (Mean ± SEM, n = 8).

| | Percent Increase in 50% MWT (g) (as compared to Day 15 of study) | | | | | |
|---|---|---|---|---|---|---|
| Group | Day 1 | Day 15 | Day 16 | Day 23 | Day 30 | Day 37 |
| Duloxetine 5 mg/kg | 0.0 | 0.7 | 52.9 | 90.3 | 108.2 | 70.9 |
| ALMB-0166 15 mg/kg | 0.0 | 1.4 | 36.5 | 28.5 | 35.9 | 13.4 |
| ALMB-016625 mg/kg | 0.0 | 1.6 | 42.5 | 67.6 | 79.9 | 63.8 |
| ALMB-0166 50 mg/kg | 0.0 | −0.7 | 47.7 | 99.5 | 168.2 | 97.7 |

When comparing to the model (no osteoarthritis) rats, a significant different in pain threshold of plantar pain was observed for all osteoarthritis model test groups (multivariate test, p<0.01, α=0.05; Table 4). Moreover, when comparing the pain threshold of plantar pain over time, a significant increase was observed for the ALMB-0166 25 mg/kg and 50 mg/kg dosage groups, and the duloxetine 5 mg/kg test groups when compared with the osteoarthritis model (vehicle-treated) test group (multivariate test, p<0.05, α=0.05; Table 4).

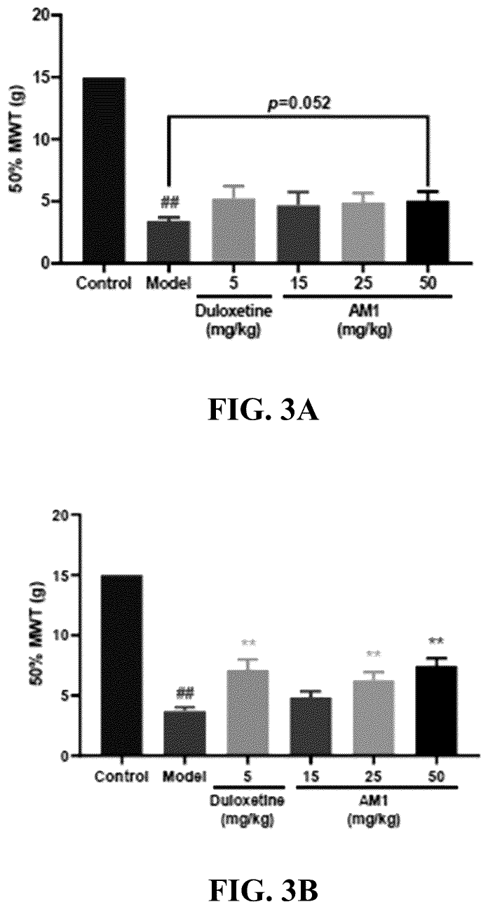

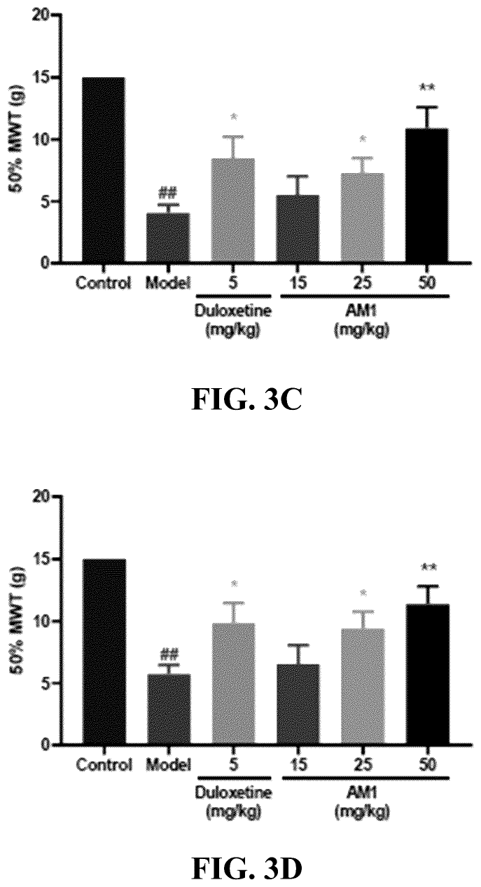

When analyzing the pain threshold of plantar pain at each time point, a significant increase in pain threshold of plantar pain was significantly increased for the ALMB-0166 25 mg/kg and 50 mg/kg. and the duloxetine dosage groups at Days 23, 30 and 37 of the study (p<0.05, α=0.05; Table 4). An increase in the pain threshold of plantar pain was also observed for the ALMB-0166 15 mg/kg dosage group over the course of treatment, but the different did not reach the significance threshold when compared to the osteoarthritis (vehicle-treated) animals (p=0.52, α=0.05; Table 4). Moreover, the increase in pain threshold of plantar pain resulting from treatment with ALMB-0166 showed a dose-dependent effect (FIGS. 3A-3D).

Effect of Anti-Cx43 Antibody on Weight Bearing Difference of Hind Legs of Rats with Osteoarthritis Induced by pMMx+ACLT

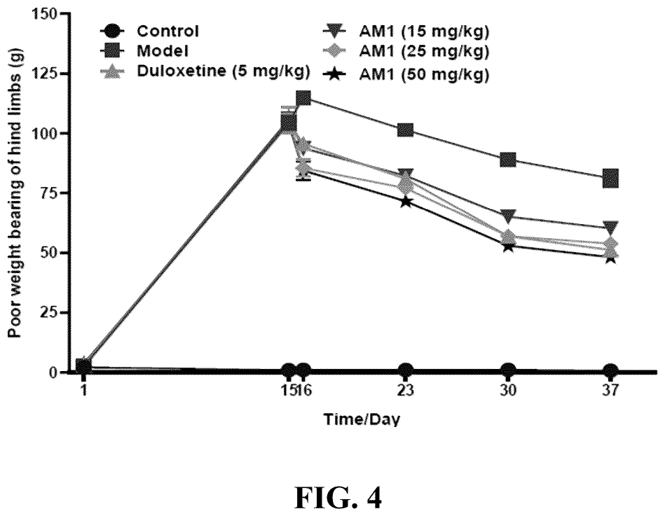

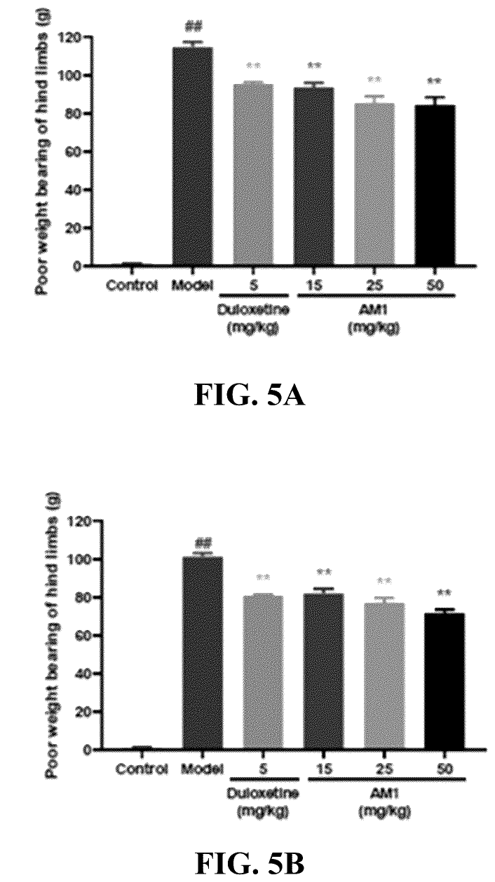

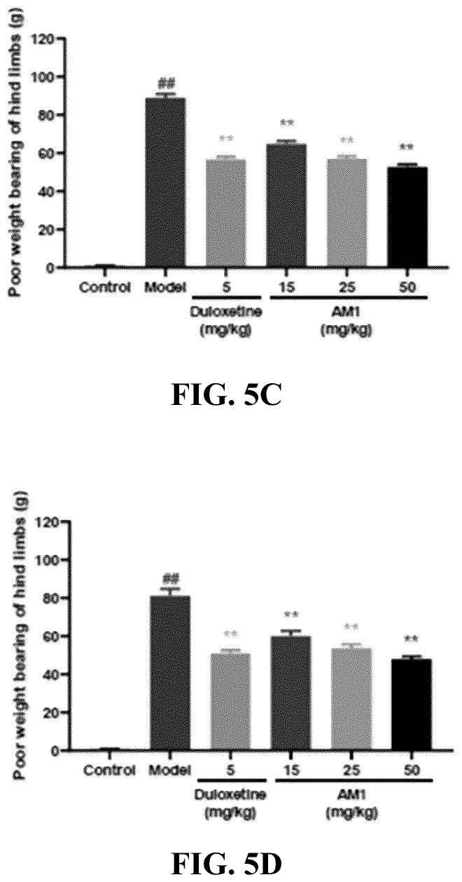

The hind limb weight bearing difference decreased over time for all osteoarthritis test groups (Table 6 and FIG. 4). The decrease in hind limb weight bearing difference was significant for the three ALMB-0166 dosage groups and the duloxetine 5 mg/kg test groups when compared with the osteoarthritis model (vehicle-treated) test group (multivariate test, p<0.05, α=0.05; Table 6). When analyzing the hind limb weight bearing at each time point, a significant decrease in the weight bearing difference was observed on Days 16, 23, 30 and 37 of the study for all ALMB-0166 dosage groups, as well as the duloxetine test group (multivariate test, p<0.01, α=0.05; Table 6). In addition, the decrease in the hind limb weight difference resulting from treatment with ALMB-0166 showed a dose-dependent effect (FIGS. 5A-5D).

TABLE 6
Weight bearing difference of hind legs of animal test groups over study period (Mean ± SEM, n = 8).

| | 50% MWT (g) | | | | | |
|---|---|---|---|---|---|---|
| Group | Day 1 | Day 15 | Day 16 | Day 23 | Day 30 | Day 37 |
| Control (no | 2.13 ± 1.16 | 0.97 ± 0.70 | 1.12 ± 0.84 | 1.05 ± 0.99 | 1.10 ± 0.68 | 0.77 ± 0.48 |
| osteoarthritis | | | | | | |
| induction) | | | | | | |
| Model | 2.48 ± 1.74 | 104.46 ± 8.12^{##} | 114.91 ± 7.03^{##} | 101.38 ± 5.08^{##} | 88.95 ± 5.89^{##} | 81.18 ± 10.35^{##} |
| (vehicle- | | | | | | |
| treated) | | | | | | |
| Duloxetine | 2.96 ± 1.88 | 104.28 ± 11.40 | 95.60 ± 2.27** | 80.91 ± 1.98 | 56.80 ± 4.06** | 51.12 ± 4.81** |
| 5 mg/kg | | | | | | |
| ALMB-0166 | 2.93 ± 2.22 | 105.47 ± 9.11 | 93.72 ± 6.68** | 82.22 ± 6.32* | 64.97 ± 3.88** | 60.14 ± 7.34** |
| 15 mg/kg | | | | | | |
| ALMB-0166 | 3.00 ± 2.11 | 105.90 ± 14.01 | 85.49 ± 9.94** | 77.07 ± 7.22** | 56.88 ± 4.19** | 53.73 ± 5.94** |
| 25 mg/kg | | | | | | |
| ALMB-0166 | 3.80 ± 2.73 | 104.01 ± 8.65 | 84.38 ± 11.57** | 71.61 ± 5.63** | 52.90 ± 3.59** | 48.09 ± 4.06** |
| 50 mg/kg | | | | | | |

Note:
^{##}P < 0.01 vs. Control;
*P < 0.05,
**P < 0.01 vs. Model

### CONCLUSIONS

Treatment of a rat model of osteoarthritis (induced by pMMX+ACLT) with anti-CX43 antibody ALMB-0166 at dosages of 15 mg/kg, 25 mg/kg and 50 mg/kg led to an improvement in the gait score over time. Moreover, treatment of the osteoarthritis model rats with anti-Cx43 antibody ALMB-0166 at dosages of 15 mg/kg, 25 mg/kg and 50 mg/kg also led to significant improvements in the mechanical pain threshold as well as in the hind limb weight bearing difference. The analgesic effect observed on osteoarthritis rats treated with the anti-Cx43 antibody ALMB-0166 was dose-dependent effect, and the analgesic effect resulting from treatment with 25 mg/kg and 50 mg/kg ALMB-0166 comparable or better than that resulting from treatment with 5 mg/kg duloxetine.

TABLE 7
Table of Sequences.

| SEQ | | |
|---|---|---|
| ID | | |
| NO | Description | Amino Acid Sequence |
| 1 | HCDR1 | GYTFTSYY |
| 2 | HCDR2 | INPSNAGT |
| 3 | HCDR3 | TREGNPYYTMNY |
| 4 | LCDR1 | QSLLNSGNQKTY |
| 5 | LCDR2 | GAS |
| 6 | LCDR3 | QNDHSYPFT |
| 7 | VH sequence | EVQLVQSGAEVKKPGASVKVSCKASGYTFTSYYMYWVRQAPGQGLEWIGGINPSNAGT |
| | | NFNEKFKNRATLTVDKSTSTAYMELSSLRSEDTAVYYCTREGNPYYTMNYWGQGTLVT |
| | | VSS |
| 8 | VL sequence | DIVMTQSPDSLAVSLGERATISCKSSQSLLNSGNQKTYLAWYQQKPGQPPKLLIYGASTRE |
| | | SGVPDRFSGSGSGTDFTLTISSLQAEDVAVYYCQNDHSYPFTFGQGTKLEIK |
| 9 | Heavy chain | EVQLVQSGAEVKKPGASVKVSCKASGYTFTSYYMYWVRQAPGQGLEWIGGINPSNaGTN |
| | of Ab # K | FNEKFKNRATLTVDKSTSTAYMELSSLRSEDTAVYYCTREGNPYYTMNYWGQGTLVTVS |
| | | SASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPEAAGGP |
| | | SVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 10 | Light chain | DIVMTQSPDSLAVSLGERATISCKSSQSLLNSGNQKTYLAWYQQKPGQPPKLLIYGASTRE |
| | | SGVPDRFSGSGSGTDFTLTISSLQAEDVAVYYCQNDHSYPFTFGQGTKLEIKRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 11 | Heavy chain | EVQLVQSGAEVKKPGASVKVSCKASGYTFTSYYMYWVRQAPGQGLEWIGGINPSNgGTN |
| | of Ab # E | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFLGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 12 | Heavy chain | EVQLVQSGAEVKKPGASVKVSCKASGYTFTSYYMYWVRQAPGQGLEWIGGINPSNaGTN |
| | of Ab # G | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFLGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 13 | Heavy chain | EVQLVQSGAEVKKPGASVKVSCKASGYTFTSYYMYWVRQAPGQGLEWIGGINPSNgGTN |
| | of Ab # I | FNEKFKNRATLTVDKSTSTAYMELSSLRSEDTAVYYCTREGNPYYTMNYWGQGTLVTVS |
| | | SASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPEAAGGP |
| | | SVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 14 | Heavy chain | EVQLVQSGAEVKKPGASVKVSCKASGYTFTSYYMYWVRQAPGQGLEWIGGINPSNgGTN |
| | of Ab # C | FNEKFKNRATLTVDKSTSTAYMELSSLRSEDTAVYYCTREGNPYYTMNYWGQGTLVTVS |
| | | SASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 15 | Heavy chain | EVQLVQSGAEVKKPGASVKVSCKASGYTFTSYYMYWVRQAPGQGLEWIGGINPSNgGTN |
| | of Ab # M | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFEGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 16 | Heavy chain | EVQLVQSGAEVKKPGASVKVSCKASGYTFTSYYMYWVRQAPGQGLEWIGGINPSNaGTN |
| | of Ab # O | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEFEGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 17 | Heavy chain | EVQLVQSGAEVKKPGASVKVSCKASGYTFTSYYMYWVRQAPGQGLEWIGGINPSNgGTN |
| | of Ab # Q | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 18 | Heavy chain | EVQLVQSGAEVKKPGASVKVSCKASGYTFTSYYMYWVRQAPGQGLEWIGGINPSNaGTN |
| | of Ab # S | FNEKFKNRATLTVDKSTSTAYMELSSLRSEDTAVYYCTREGNPYYTMNYWGQGTLVTVS |
| | | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 19 | Epitope | FLSRPTEKTI |

The invention claimed is:

1. A method for treating osteoarthritis in a subject in need thereof, comprising administering to the subject at least one dose of an anti-connexin 43 (Cx43) antibody, wherein the anti-Cx43 antibody comprises heavy chain CDR sequences and light chain CDR sequences as follows:
HCDR1: SEQ ID NO: 1;
HCDR2: SEQ ID NO: 2;
HCDR3: SEQ ID NO: 3;
LCDR1: SEQ ID NO: 4;
LCDR2: SEQ ID NO: 5;
and
LCDR3: SEQ ID NO: 6.

2. The method of claim 1, comprising administering a first dose of the anti-Cx43 antibody at day 1, and a second dose at day 8, optionally further comprising administering a third dose of the anti-Cx43 antibody at day 15, optionally further comprising administering a fourth dose of the anti-Cx43 antibody at day 22, optionally further comprising administering subsequently doses weekly after the fourth dose.

3.-5. (canceled)

6. The method of claim 1, wherein the at least one dose of the anti-Cx43 antibody is about 0.01 mg/kg to about 100 mg/kg, optionally wherein the at least one dose of the anti-Cx43 antibody is about 15 mg/kg, about 25 mg/kg, or about 50 mg/kg.

7.-9. (canceled)

10. The method of claim 1, wherein the anti-Cx43 antibody is administered intravenously, and/or the subject is a human.

11. The method of claim 1, wherein at least one indicator of osteoarthritis severity is assessed at least one day to one week after each dose is administered,
optionally wherein the at least one indicator of osteoarthritis severity comprises:
(a) pain threshold of the arthritis,
(b) gait score, and/or
(c) weight bearing difference.

12. (canceled)

13. The method of claim 11, wherein the at least one indicator of osteoarthritis severity is improved after at least one dose of the anti-Cx43 antibody is administered.

14. (canceled)

15. The method of claim 1, wherein the anti-Cx43 antibody comprises heavy chain variable sequence of SEQ ID NO: 7, and/or light chain variable sequence of SEQ ID NO: 8.

16. The method of claim 1, wherein the anti-Cx43 antibody comprises heavy chain sequence of any one of SEQ ID NOs: 9, 12, 16 and 1841-18, and/or light chain variable sequence of SEQ ID NO: 10.

17. The method of claim 16, wherein the anti-Cx43 antibody comprises heavy chain sequence of SEQ ID NO: 9, and/or light chain variable sequence of SEQ ID NO: 10.

18. A method for treating osteoarthritis in a subject in need thereof, comprising administering to the subject an effective amount of an anti-connexin 43 (Cx43) antibody, wherein the anti-Cx43 antibody is administered according to the following dosage regimen:
i) a first dose at day 1;
ii) a second dose at day 8;
iii) a third dose at day 15; and
iv) a fourth dose at day 21; or
the method optionally further comprises administering subsequent doses of the anti-Cx43 antibody weekly after the fourth dose.

19. (canceled)

20. The method of claim 18, wherein the first, second, third, fourth and/or subsequent doses are about 0.01 mg/kg to about 100 mg/kg, optionally wherein the first, second, third, fourth and/or subsequent doses of the anti-Cx43 antibody are about 15 mg/kg, about 25 mg/kg, or about 50 mg/kg.

21.-23. (canceled)

24. The method of claim 18, wherein the anti-Cx43 antibody administered intravenously, and/or wherein the subject is a human.

25. The method of claim 18, wherein at least one indicator of osteoarthritis severity is assessed at least one day to one week after each dose is administered;
optionally wherein the at least one indicator of osteoarthritis severity comprises (a) pain threshold of the arthritis, (b) gait score, and/or (c) weight bearing difference.

26. (canceled)

27. The method of claim 25, wherein the at least one indicator of osteoarthritis severity is improved after at least one dose of the anti-Cx43 antibody is administered.

28. (canceled)

29. The method of claim 18, wherein the anti-Cx43 antibody comprises heavy chain CDR sequences and light chain sequences as follows:
HCDR1: SEQ ID NO: 1;
HCDR2: SEQ ID NO: 2;
HCDR3: SEQ ID NO: 3;
[and]]
LCDR1: SEQ ID NO: 4;
LCDR2: SEQ ID NO: 5;
and
LCDR3: SEQ ID NO: 6.

30. The method of claim 18, wherein the anti-Cx43 antibody comprises heavy chain variable sequence of SEQ ID NO: 7, and/or light chain variable sequence of SEQ ID NO: 8.

31. The method of claim 18, wherein the anti-Cx43 antibody comprises heavy chain sequence of any one of SEQ ID NOs: 9 and 11-18, and/or light chain variable sequence of SEQ ID NO: 10.

32. The method of claim 31, wherein the anti-Cx43 antibody comprises heavy chain sequence of SEQ ID NO: 9, and/or light chain variable sequence of SEQ ID NO: 10.

33. The method of claim 1, wherein the anti-Cx43 antibody blocks the opening of Cx43 hemichannel in the subject.

34. The method of claim 18, wherein the anti-Cx43 antibody blocks the opening of Cx43 hemichannel in the subject.
